(19) United States
(12) Patent Application Publication
(10) Pub. No.: US 2018/0042836 A1
(43) Pub. Date: Feb. 15, 2018

(54) DELIVERY OF ORAL CARE SUBSTANCES

(71) Applicant: KONINKLIJKE PHILIPS N.V., EINDHOVEN (NL)

(72) Inventors: MICHIEL JOHANNES JONGERIUS, EINDHOVEN (NL); EMILE JOHANNES KAREL VERSTEGEN, EINDHOVEN (NL); NICOLAAS PETRUS WILLARD, VALKENSWAARD (NL); CHRISTIANNE ROSETTE MARIA DE WITZ, LOMMEL (BE); VEENA MOHAN, CAMBRIDGE (GB)

(73) Assignee: KONINKLIJKE PHILIPS N.V., EINDHOVEN (NL)

(21) Appl. No.: 15/556,034

(22) PCT Filed: Mar. 4, 2016
(86) PCT No.: PCT/EP2016/054651

§ 371 Date: Sep. 6, 2017

(30) Foreign Application Priority Data
Mar. 6, 2015 (EP) 15158015.6

(51) Int. Cl. A61K 8/899; A61K 8/22; A61C 19/06; A61Q 11/00

(52) U.S. Cl. CPC A61K 8/899; A61Q 11/00; A61K 8/22; A61C 19/066; A61K 2800/92; A61K 2800/95; A61K 2800/87

(57) ABSTRACT

Disclosed is a hydrophilic silicone composition comprising an oral care substance. The composition is based on polysiloxanes having a high water-uptake capability. An aqueous solution or dispersion of the oral care substance can be soaked into the polysiloxane, whereby the resulting composition shows favorable release characteristics. The use of hydrophilically modified polysiloxanes, possibly in conjunction with unmodified hydrophobic polysiloxanes, allows for the convenient production of desired shapes, advantageously based on a dental imprint. The hydrophilic modification of the polysiloxane is particularly by including a hydrophilic vinyl monomer, particularly an alpha-olefinic soap, into the composition prior to curing.

### FIELD OF THE INVENTION

The invention is in the field of compositions for the delivery of oral care agents to the oral cavity. Particularly, the invention pertains to compositions based on silicone for the sustained release of oral care agents, more particularly dental bleaching agents.

### BACKGROUND OF THE INVENTION

The human oral cavity, notably teeth and gums, is generally in need of oral care agents. Think of, e.g., antiplaque agents, anti-tartar agents, anti-gingivitis agents, anti-bacterial agents, and others.

Such agents are generally administered from toothpastes and/or oral rinse liquids. Due to the typical environment of the oral cavity, e.g. having saliva present, a standard difficulty in the art is that active agents from toothpastes and oral rinses are quickly reducing in concentration after their application. Therefore they cannot protect the mouth for long times, and they need therefore to be applied several times daily.

It is desired in the field to present compositions that provide a desirable substantivity. I.e., by which actives can be maintained on the hard and soft oral tissues for a period of time sufficient to enhance or prolong the therapeutic, prophylactic, and/or cosmetic benefits provided by the actives. Particularly, it is desired to provide compositions for the sustained release of oral care agents. To this end, materials are sought that are acceptable for use in the oral cavity, and which are capable to store and then release oral care agents therefrom at a sustained slow rate.

WO 2007/083253 discloses compositions for delivering oral care substances to the oral cavity, based on silicone pressure sensitive adhesives. The composition comprises, in addition to the silicone pressure sensitive adhesive, inter alia, a plasticizing material and a water-soluble bioadhesive polymer. Thus, several components need to be combined in order to provide a composition having the desired storage and release properties. It is desired to provide a simpler system, particularly with improved storage and/or release characteristics.

Another desire in the art is to provide a system which allows the release of an oral care active substance from one side (applied towards teeth) and protection from leakage to the aqueous outside environment (saliva) from the other sides. To this end protective layers may be applied. However, the need to apply a protective layer complicates the manufacturing process in the event that shaped devices are to be provided. Not all protective materials lend themselves to being included in molding, and after preparing the shaped device, the choice of protective materials is limited to materials that can be applied so as to sufficiently tightly follow the already formed shape.

Further, it would be desired to provide a material from which oral care agents can be released, and which can be shaped as a dental impression.

As background art, reference is further made to US 2014/031734. This document concerns a silicone adhesive composition including an ionic silicone, which is disclosed to be useful for healthcare applications such as wound care and drug delivery. Amongst many suggested types of drugs, also oral care drugs are mentioned. The document does not specifically teach a silicone adhesive composition for local delivery of oral care agents. Particularly, the document does not teach the delivery of dental bleaching agents. These agents come with specific challenges, due to the relatively high amount of bleaching agent that needs to be administered.

### SUMMARY OF THE INVENTION

In order to better address the above and other desires, the invention provides, in one aspect, a composition for delivering an oral care substance to the oral cavity, said composition comprising at least one oral care agent contained in an elastomeric polysiloxane matrix comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers.

In another aspect, the invention presents the use of an elastomeric polysiloxane comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers, as a carrier for an oral care substance.

In yet another aspect, the invention resides in a method for delivering an oral care substance to the oral cavity, said substance being administered to the oral cavity in a polymer matrix, wherein the polymer is an elastomeric polysiloxane comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers.

In a still further aspect, the invention provides a shaped article comprising a layer of hydrophobic polysiloxane elastomer molded against a layer of a hydrophilic polysiloxane elastomer, said hydrophilic polysiloxane comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers, wherein the hydrophobic polysiloxane determines one outer surface of the device and the hydrophilic polysiloxane determines another outer surface of the device.

In yet another aspect, the invention presents a dental tray comprising a material shaped according to a dental impression and comprising an oral care active substance to be released therefrom, wherein said material comprises hydrophilic polysiloxane comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers.

In a still further aspect, the invention is a kit for the local administration of an oral care agent to the teeth of a subject, the kit comprising

a. a dental tray made of a silicone elastomer;
b. a curable hydrophilic silicone material;
c. an aqueous liquid comprising an oral care agent;
wherein the curable hydrophilic silicone material comprises a siloxane prepolymer component comprising vinyl groups and a siloxane crosslinker component comprising Si—H groups, wherein either or both of the components comprise an alpha-olefinic soap.

In another aspect, the invention is a method of making a composition for delivering an oral care substance to the oral cavity, the method comprising the steps of

(i) providing a hydrophilic polysiloxane capable of taking up more than 10% by weight of water and up to 500% by weight of water, after immersion in demineralized water at room temperature for a sufficient time to reach saturation;
(ii) providing an aqueous liquid comprising an oral care agent dissolved or dispersed therein;
(iii) soaking the aqueous liquid into the hydrophilic polysiloxane.

### BRIEF DESCRIPTION OF THE DRAWINGS

FIG. 1 to FIG. 9 show various graphs relating to test results. The figures are discussed in the examples.

### DETAILED DESCRIPTION OF THE INVENTION

The hydrophilic polysiloxane (hydrophilic silicone) matrix used in the present invention is a material that is capable of taking up very high amounts of water, as discussed below. In a broad sense, the invention is based on the judicious insight to provide, as a novel use of such a material, compositions for delivering an oral care substance to the oral cavity. Thereby a high water-uptake hydrophilic siloxane polymer is employed as a matrix for an oral care substance, particularly for a hydrophilic oral care substance.

According to the invention, it has been found that hydrophilic silicone material as created according to the methods of WO 2013/001506 and WO 2013/001487 has the unexpected capability for uptake, storage and release of agents in its bulk volume. This provides the benefit that significant amounts of oral care agents can be stored in the silicone. The transport rates by diffusion are relatively low (a typical diffusion coefficient D being of the order of 10⁻¹² m²/s), so that a continuous sustained release of agents during a long time period (hours to days) can be maintained.

This is of particular interest, although not limited thereto, for hydrogen-peroxide or other bleaching agents (for tooth whitening, and for anti-bacterial applications, e.g. halitosis), fluoride agents (for tooth strengthening), and sensitivity-reducing agents such as amorphous calcium phosphate (ACP).

The polymers suitable for use in the present invention are hydrophilic siloxane polymers as described in WO 2013/001487 A1. These are rubbery or elastomeric polymer materials taking up more than 5% by weight of water and up to 500% by weight of water, after immersion in demineralized water at room temperature for a sufficient time to reach saturation. Particularly, the compositions used in the invention are capable of taking up more than 40% by weight of water, and up to 500% by weight, or up to 200%, or 250%) by weight, or up to 120% by weight, of water after immersion in demineralized water at room temperature for a sufficient time such as 5 days or more to reach saturation.

Polymerized siloxanes or polysiloxanes, also known as silicones, are mixed inorganic-organic polymers with the chemical formula [R₂SiO]ₙ, where R is an organic group such as alkyl (e.g., methyl, ethyl) or aryl (e.g., phenyl). These materials consist of an inorganic silicon-oxygen backbone chain ( . . . —Si—O—Si—O—Si—O— . . . ) with organic side groups attached to the silicon atoms.

The polysiloxanes used in the present invention comprise two different types of units based on siloxane monomers. One type is formed by repeating units of hydrophobic siloxane monomers. This refers to such units as do normally form the basis of polysiloxanes, with a typical example being repeating units of dimethyl siloxane. In general, these units are formed of dialkyl siloxane, diaryl siloxane, or mixtures thereof. Thereby the alkyl groups, each independently, preferably have a chain length of up to six carbon atoms (C₆), more preferably C₁₋₄ and most preferably methyl. The aryl groups are, e.g., phenyl, substituted phenyl such as mono- or dialkyl (with alkyl having the aforementioned meaning), pyridyl, naphtyl, or combinations thereof preferably, aryl is phenyl.

The invention particularly pertains to the type of elastomeric polysiloxanes (also known as silicone rubbers) produced by the crosslinking of two different types of siloxane precursor components. One is a siloxane component (generally viewed as a prepolymer) comprising vinyl groups. The other is a siloxane component (generally viewed as a crosslinker) comprising Si—H groups. Therefrom a silicone rubber (elastomer) is generally formed by the platinum salt induced crosslinking of said components.

The aforementioned components are generally hydrophobic. The hydrophilic siloxane elastomers used in the present invention are prepared by adding, to the hydrophobic siloxane components that are normally applied to make up an elastomeric polysiloxane matrix, a compound that comprises an ethylenically unsaturated group, so as to be reactive in the crosslinking reaction in which the vinyl groups-containing siloxane prepolymer participates, and a hydrophilic group, i.e., a hydrophilic vinyl monomer. In order for said hydrophilic vinyl monomer to be compatible with the hydrophobic polysiloxane, the monomer is an alpha-olefinic soap, i.e., an amphiphilic molecule comprising a hydrophilic and a hydrophobic end (typically a polar head and an apolar tail).

The vinyl group, i.e., a terminal, ethylenically unsaturated (alpha-olefinic) group present in the hydrophilic vinyl monomer refers, inter alia, to vinyl containing hydrophilic molecules, for example an ethylenically unsaturated (olefinic) soap, particularly an alpha-olefinic soap such as an alpha-olefinic sulfonic acid sodium salt. Other, possibly additional suitable olefinic groups for the hydrophilic alkene monomer are, e.g., allyl, acrylic or methacrylic groups.

A preferred hydrophilic monomer is an alpha-olefin or alkenyl sulfonate having 3 to 28 (preferably 10 to 18, more preferably 12 to 16) carbon atoms in association with a cation. Said cation may be a monovalent cation selected from the group consisting of ammonium and alkali metal cations (such as, but not limited to, the cations of Li, Na, or K). Said cation may also be a divalent cation selected from the group consisting of alkaline-earth metal cations (such as the cations of Ca or Mg). Other hydrophilic side groups can also comprise at least one moiety from ionic groups such as sulphate, phosphate, phosphonate, carboxylate, ammonium, or phosphonium, or combinations of these groups like in betaine or sulfobetaine. It can also contain non ionic hydrophilic groups like alcohol groups such as hydroxy, glycols, sugar derivatives, ethers such as glycol ether, amines, amides, phosphine oxide, aldehydes or esters. Preferred counter ions comprise the before mentioned ammonium, alkali, earth alkali ions, H⁺ or mixtures thereof. For positive hydrophilic side chains the preferred counter ions are the halogenides, hydroxide, acetate, sulphite, sulphate, nitrite, nitrate, phosphate, perchlorate, tetrafluoroborate, or mixtures thereof.

Preferred hydrophilic monomers are sodium alpha-olefin sulfonates (preferably sodium C₁₂₋₁₄ olefin sulfonate). Commercially available from Stepan Company (Northfield, Ill., United States), these are mixtures of long-chain sulfonate salts prepared by the sulfonation of alpha olefins. Based on approval in the USA by the Food and Drug Administration (FDA) the ammonium, calcium magnesium, potassium and sodium salts of the alpha-olefin sulfonates are preferred.

The hydrophilic monomer, i.e. particularly an alpha-olefinic soap, can directly be added to the mixture of polysiloxane precursor components. At least in part it will thus be incorporated into the silicone matrix by the aforementioned cross-linking reaction, and in part they can be present just mixed into the silicone network. It can also be reacted with one of the polysiloxane precursor components prior to the mixing of these precursor components. Also, it is conceivable to mix all of the siloxane monomers prior to the formation of prepolymers, and thereby include the hydrophilic monomer. Combinations of these embodiments can also be made. Preferably, the composition of the invention comprises one or more unreacted alpha-olefinic soap monomers.

In one embodiment of the present invention, the total number of hydrophobic siloxane repeating units (a) and hydrophilic repeating units (b) is at least 5 and less than 1,000. Generally, the molar ratio of the repeating units (a) to the repeating units (b) is at least 4.5, more preferably at least 7, more preferably at least 9, most preferably at least 13. Generally the molar ratio of the repeating units (a) to the repeating units (b) is at most 90, preferably at most preferably 40, most preferably at most 25.

Preferably, e.g. for the purpose of an increased water-uptake, the total weight of alpha-olefinic soap monomers in the composition is 10-30% relative to the total weight of the siloxane monomers in the composition.

The elastomeric polysiloxane used in the present invention has an exceptionally high water uptake capacity. For instance it takes up well above 25% by weight, and more typically more than 40% by weight of water after immersion in demineralized water at room temperature for a sufficient time such as 5 days or more to reach saturation. The elastomeric polysiloxane used in the invention will generally take up at most 500% by weight (for instance at most 250%) by weight, or at most 200%, such as at most 120% by weight, or at most 50% by weight, such as at most 40% by weight of water after immersion in demineralized water at room temperature for a sufficient time such as 5 days or more to reach saturation.

The compositions according to the invention can be prepared in accordance with the teaching in WO 2013/001487. The invention presents a novel use of these compositions. Particularly, in one aspect, the invention includes the use of an elastomeric polysiloxane comprising a network based on repeating units of hydrophobic siloxane monomers and units of siloxane monomers comprising one or more hydrophilic side groups, as a carrier for an oral care substance.

The oral care active substance can be added to the hydrophilic polysiloxane composition as a solution or dispersion in water, by being soaked into the hydrophilic polysiloxane (e.g. by immersion).

The invention is suitable for use with oral care agents that are capable of being dissolved, emulsified, or suspended in an aqueous liquid that can be taken up by the hydrophilic polysiloxane. This particularly pertains to hydrophilic oral care agents. Such agents are either present in the form of aqueous solutions, or in the form of hydrophilic particles.

Representative examples of such aqueous solutions include solutions of hydrogen peroxide in water, in various possible different concentrations. Representative examples of hydrophilic particles include solid peroxides, such as carbamide peroxide and polyvinylpyrrolidone hydrogenperoxide adduct. Examples of peroxides for use as whitening agents (also known as “bleaching agents”) in the hydrophilic polysiloxane composition of the invention include, but are not limited to, hydrogen peroxide, carbamide peroxide, polyvinylpyrrolidone hydrogenperoxide adduct and other hydrogen peroxide complexes, alkali metal percarbonates, perborates, such as sodium perborate, persulfates, such as potassium persulfate, calcium peroxide, zinc peroxide, magnesium peroxide, strontium peroxide, peroxyacids, and combinations thereof use is made of an aqueous, or otherwise polar, liquid or solid comprising a peroxide. Non-limiting examples thereof include a solution of hydrogen peroxide in water, carbamide peroxide, or complexes of PVP and hydrogen peroxide.

Preferred are bleaching (whitening) agents as discussed above. Other hydrophilic agents suitable for use in the compositions of the invention are, e.g., selected from the group consisting of remineralizing agents, anti-caries agents, tartar control (anticalculus) agents, abrasives, anti-plaque agents, anti-odor agents, fluoride agents, anti-bacterial agents, biofilm preventing or dispersing agents, antioxidants, pH regulating agents, long-term protective components, reactive enzymes, reactive radicals, anti-inflammatory agents, coloring agents, such as titanium dioxide, flavoring agents, coloring agents, dyes, particles and combinations thereof. In an interesting embodiment, one or more of the aforementioned agents are present in addition to whitening agents.

Specific examples of oral care agents include:

Tartar control (anticalculus) agents: these may include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts, and mixtures thereof.

Fluoride ion sources: These may be useful, for example, as an anti-caries agent. Orally acceptable fluoride ion source which can be used include potassium, sodium and ammonium fluorides and monofluorophosphates, stannous fluoride, indium fluoride and mixtures thereof.

Tooth and soft tissue desensitizers: these may include stannous ions, such as halides and carboxylate salts, arginine, potassium citrate, potassium chloride, potassium tartrate, potassium bicarbonate, potassium oxalate, potassium nitrate, strontium salts, and mixtures thereof.

Antimicrobial (e.g., antibacterial) agents: these may include orally acceptable antimicrobial agents, such as Triclosan (5-chloro-2-(2,4-dichlorophenoxy)phenol); 8-hydroxyquinoline and salts thereof, zinc and stannous ion sources such as zinc citrate; copper (II) compounds such as copper (II) chloride, fluoride, sulphate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride); bisguanides, such as chlorhexidine digluconate; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; grapeseed extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; and mixtures thereof. Other useful antimicrobials are disclosed in U.S. Pat. No. 5,776,435.

Antioxidants: orally acceptable antioxidants which can be used include butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Antiplaque (e.g., plaque disrupting) agent: orally acceptable antiplaque agents can include stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates, and mixtures thereof.

Anti-caries agents: examples of these include calcium glycerylphosphate and sodium trimetaphosphate.

Anti-inflammatory agents: orally acceptable anti-inflammatory agents can include steroidal agents, such as flucinolone and hydrocortisone, and nonsteroidal agents (NSAIDs) such as ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone, and mixtures thereof.

H₂ antagonists: antagonists useful herein include cimetidine, etintidine, ranitidine, ICIA-5165, tiotidine, ORF-17578, lupititidine, donetidine, famotidine, roxatidine, pifatidine, lamtidine, BL-6548, BMY-25271, zaltidine, nizatidine, mifentidine, BMY-52368, SKF-94482, BL-6341A, ICI-162846, ramixotidine, Wy-45727, SR-58042, BMY-25405, loxtidine, DA-4634, bisfentidine, sufotidine, ebrotidine, HE-30-256, D-16637, FRG-8813, FRG-8701, impromidine, L-643728, HB-408.4, and mixtures thereof.

Nutrients: Suitable nutrients include vitamins, minerals, amino acids, proteins, and mixtures thereof.

The oral care agent will be present in the composition in an amount generally ranging from 0.1 wt. % to 50 wt. % preferably 1 wt. % to 25 wt. %.

As examples of other additives, the hydrophilic polysiloxane composition may include one or more of the following:

Colorants: The colorant may be selected to provide the film with a white appearance or a tint, which are stable and are not affected by oxidation reactions up to about 0.5% coloring agent and/or dyes. These coloring agents and/or dyes include titanium dioxide or Food, Drug and Cosmetic (FD&C) colorants including primary FD&C, Blue No. 1, FD&C Blue No. 2, FD&C Green No. 3, FD&C yellow No. 5, FD&C Yellow No. 6, FD&C Red No. 3, and FD&C Red No. 40 and lakes FD&C Blue No. 1, FD&C Blue No. 2, FD&C Yellow No. 5, FD&C Yellow No. 6, FD&C Red No. 2, FD&C Red No. 3, FD&C Red No. 40 as disclosed in US 2002/0197215 A1 and combinations thereof.

Tooth and soft tissue desensitizers: these may include stannous ions, such as halides and carboxylate salts, arginine, potassium citrate, potassium chloride, potassium tartrate, potassium bicarbonate, potassium oxalate, potassium nitrate, strontium salts, and mixtures thereof.

Anti-staining agents: such as fluorine-based compound or a silicone-based compound e.g. silicone polymers. as disclosed in patent US 20050112365 A1.

Flavoring agents: any of the flavoring agents commonly used in toothpastes may be used, by way of example.

Other possible additives include thermochromics. These types of additives may enable a user to visually determine when a treatment is complete.

The additional agents and additives, if present, can be present in amounts generally known to the skilled person. A typical range is of from 0.5 wt. % to 10 wt. %, particularly of from 2 wt. % to 5 wt. %.

The invention also pertains to a method for delivering an oral care substance, as described above, to the oral cavity of a subject, preferably a human subject. Thereby said substance is administered to the oral cavity comprised in a polymer matrix. Therein the polymer is an elastomeric polysiloxane comprising a network based on repeating units of hydrophobic siloxane monomers and units of siloxane monomers comprising one or more hydrophilic side groups. Preferably, the polymer is as described in any one of the above-described embodiments.

Particularly usefully, the invention allows providing a shaped article which, on one side thereof, is capable of releasing an oral care active substance and, on another side thereof, provides a protective layer. To this end, the device comprises a layer of hydrophobic polysiloxane elastomer molded against a layer of a hydrophilic polysiloxane elastomer. The hydrophilic polysiloxane comprises a network based on repeating units of hydrophobic siloxane monomers and units of siloxane monomers comprising one or more hydrophilic side groups. The hydrophilic polysiloxane is preferably as described in any one of the above-described embodiments. The hydrophobic polysiloxane can be any conventional silicone elastomer. Preferably, the hydrophobic polysiloxane is the same type of polysiloxane as the hydrophilic polysiloxane, but for the modification with hydrophilic groups. The hydrophobic polysiloxane determines one outer surface of the device and the hydrophilic polysiloxane determines another outer surface of the device. The device can be, e.g., a sheet consisting of two layers on top of each other, one layer being made up of the hydrophobic polysiloxane, the other of the hydrophilic polysiloxane. The hydrophilic layer can be provided with an oral care active substance by soaking an appropriate aqueous liquid into it, at described above. It is conceivable to have other layers (silicone or different materials) present between the hydrophobic and hydrophilic polysiloxane layers as desired. However, this aspect of the invention will provide the greatest benefits in the event that use can be made of the fundamental similarity between the two polysiloxane layers. To this end, a hydrophobic and a hydrophilic polysiloxane can be molded against each other. Preferably, thereby either or both of the compositions is in a still curable state. It will be understood that the compositions, when molding, are to be in a substantially non-flowable state, so as to prevent inadvertent mixing of the two layers.

In connection with the foregoing, the invention also presents an advantageous dental tray. This tray comprises a material shaped according to a dental impression and comprises an oral care active substance to be released therefrom. The material shaped according to a dental impression comprises hydrophilic polysiloxane comprising a network based on repeating units of hydrophobic siloxane monomers and units of siloxane monomers comprising one or more hydrophilic side groups. The dental impression material is preferably made according to any one of the embodiments described above for the hydrophilic polysiloxane.

Thus, the invention provides a composition from which an oral care composition can be released to teeth, in the form of a material that allows to be shaped according to a dental impression. This is advantageous in that the composition can thus be optimized for dental delivery for any given subject (preferably a human subject). The invention thereby presents the particular advantage that, once shaped, the composition can be loaded with the active substance in the aforedescribed simple manner, viz., by soaking an aqueous liquid comprising the substance into the hydrophilic polysiloxane.

In connection herewith, the invention also provides a kit for the local administration of an oral care agent to the teeth of a subject. The kit comprises

(a) a dental tray made of a silicone elastomer;
(b) a curable hydrophilic silicone material;
(c) an aqueous liquid comprising an oral care agent;
wherein the curable hydrophilic silicone material comprises a siloxane prepolymer component comprising vinyl groups and a siloxane crosslinker component comprising Si—H groups, wherein either or both of the components comprise an alpha-olefinic soap.

It will be understood that, as a result of the use of curable polysiloxane materials, any other desired shape can be provided other than a tray or a sheet, i.e., a shape can be provided that is adapted to only covering one or more parts of the teeth.

While the invention has been illustrated and described in detail in the drawings and foregoing description, such illustration and description are to be considered illustrative or exemplary and not restrictive; the invention is not limited to the disclosed embodiments. For example, it is possible to operate the invention in an embodiment wherein one or more oral care agents are present before curing the hydrophilic polysiloxane, and one or more other oral care agents are soaked into the composition as described hereinbefore.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain features of the invention are recited in mutually different dependent claims does not indicate that a combination of these features cannot be used to advantage. Any reference signs in the claims should not be construed as limiting the scope.

In sum the invention relates to, inter alia, a hydrophilic silicone composition comprising an oral care substance. The composition is based on polysiloxanes having a high water-uptake capability. An aqueous solution or dispersion of the oral care substance can be soaked into the polysiloxane, whereby the resulting composition shows favorable release characteristics. The use of hydrophilically modified polysiloxanes, possibly in conjunction with unmodified hydrophobic polysiloxanes, allows for the convenient production of desired shapes, advantageously based on a dental imprint. The hydrophilic modification of the polysiloxane is particularly by including a hydrophilic vinyl monomer, particularly an alpha-olefinic soap, into the composition prior to curing.

The invention will be illustrated hereinafter with reference to the following non-limiting examples.

### EXAMPLES OF MATERIALS FORMULATIONS

### Example 1: Hydrophobic Silicone Sample Making

Test samples were processed from Wacker silicones. Hydrophobic silicone sheets and trays were processed from Elastosil LR3004 with a shore 40 hardness and Elastosil LR3003 with a shore 5 hardness. The Elastosil LR materials are two-component liquid silicone rubbers. They are mixed prior to use in a 1 to 1 ratio. Mixing was performed at room temperature. Compression molding was used to realize sheets and trays.

### Example 2: Hydrophilic Silicones Sheet Samples of Shore 40 Material

The commercial silicone elastomer Elastosil LR 3004/40 was used as silicone precursor material for creating hydrophilic silicone. The silicone precursor material is a two-component system that was mixed in a 1:1 weight ratio of two components A and B. The A component consists of a silicone pre-polymer bearing reactive vinyl groups and a platinum catalyst. The B component consists of a silicone pre-polymer bearing reactive vinyl groups and a pre-polymer bearing Si—H groups. The commercial sodium alpha-olefin sulfonate (n=12-14) from Stepan Company was used for creating the hydrophilic properties in the silicone.

### 27% Soap Silicone

The manufacturing process was a follows: 12 g of the very fine sulfonate powder was mixed with 7 g of an ethanol/water mixture (50/50% by volume). Then, 19 g of the A component of the silicone precursor material was added, and mixing was carried out with a speed mixer. The ethanol was removed in the speed mixer under vacuum. Then, the silicone precursor B component (26.1 g) was added and the obtained composition was mixed. The sodium alpha-olefin sulfonate is thus covering 26.6% wt. % of the silicone precursor (A+B) weight (wt. %=weight sodium alpha-olefin sulfonate/weight silicone A+B)*100). The resulting mixing ratio of this system for component A to B was 1 to 1.37. Hydrophilic sheet material samples of thickness 0.1 or 0.5 mm thickness were prepared by pressure molding for 15 min at 140° C. Aluminum molds with a smooth surface and 0.1 and 0.5 mm thickness were used. A force of 8 tons was applied dependent on the surface of the mold. The molding pressure was 700 psi.

### 10% Soap Silicone

The manufacturing process was a follows: 4 g of sulfonate powder was mixed with 4.4 g of an ethanol water mixture (50/50% by volume). Then, 19 g of the A component of the silicone precursor material added and mixing was carried out with a speed mixer. The ethanol was removed in the speed mixer under vacuum. Then, silicone precursor B component (21.6 g) was added and the obtained composition was mixed. The commercial sodium alpha-olefin sulfonate added to the silicone precursors A+B, is thus covering 9.9% wt. % of silicone precursor (A+B) weight. The mixing ratio of this system for component A to B was 1 to 1.14. Material sheet samples were prepared by pressure molding for 15 min at 140° C.

### 3% Soap Silicone

The manufacturing process was a follows: 1.2 g of sulfonate powder was mixed with 0.4 g of an ethanol water mixture (50/50% by volume) Then, 20 g of the A component of the silicone precursor material added and mixing was carried out with a speed mixer. The ethanol was removed in the speed mixer under vacuum. Then, silicone precursor B component (20.6 g) was added and the obtained composition was mixed. The commercial sodium alpha-olefin sulfonate added to the silicone precursors A+B, is thus covering 3% wt. % of silicone precursor (A+B) weight. The mixing ratio of this system for component A to B was 1 to 1.03. Material samples were prepared by pressure molding for 15 min at 140° C.

### Example 3: Hydrophilic Silicones Sheet Samples of Shore 5 Material

The commercial silicone elastomer Elastosil LR 3003/05 for 5 Shore silicone was used as silicone precursor material. The silicone precursor material is a two component system that was mixed in a 1:1 weight ratio of two components A and B. The A component consist of a silicone pre-polymer bearing reactive vinyl groups and a platinum catalyst. The B component consists of a silicone pre-polymer bearing reactive vinyl groups and a pre-polymer bearing Si—H groups. The commercial sodium alpha-olefin sulfonate (n=12-14) from Stepan Company was used. 12 g of this very fine powder was mixed with 7 g of an ethanol water mixture (50/50% by volume). Then, 19 g of the A component of the silicone precursor material added and mixing was carried out with a speed mixer. The ethanol was removed in the speed mixer under vacuum until a small amount (0.5 gram) of water was still present. Then, silicone precursor B component (24.7 g) was added and the obtained composition was mixed. The commercial sodium alpha-olefin sulfonate added to the silicone precursors A+B, is thus covering 27.5% wt. % of silicone precursor (A+B) weight. The mixing ratio of this system for component A to B was 1 to 1.3. Material samples were prepared by pressure molding for 15 min at 140° C. at 711 psi or 4902 kPa.

### Example 4: Testing Samples

### Water Uptake and Release Testing Method.

Different amounts of Sodium-α-olefin sulfonate C₁₄-C₁₆ (from Stepan) have been incorporated in the commercial elastomer Elastosil LR 3004 with a shore value of 40 or Elastosil LR 3003 with a shore A 5 hardness. The water uptake of the cured sheets, which have a smooth surface texture (0.2-0.5 μm Ra) have been tested by immersion of a hydrophilic or hydrophobic test sheet with a known surface of about 4.4 cm² in demineralized water for a period of time (total surface is 10.2 cm²). The weight of the sheets was measured with an analytical scale and the sheets were individually put in a plastic container filled with the same amount of demineralized water (20 ml) and were measured again after 20, 60, 120, 420, 7200 minutes or variations within this timeframe. These measurements were done in duplicate.

Before the water or the 6% hydrogen-peroxide/water absorption into the hydrophobic or hydrophilic sheets are measured, the sheet samples are first dried in an oven at 120° C. for 1 hour, after which they are immediately weighed to provide a base line. The immersed samples are removed at regular intervals for these measurements. The excess liquid is first removed with absorbing. After this weighing is completed, the samples are re-immersed in the water or 6% hydrogen peroxide/water.

The release of moisture from the pre-immersed sheets was measured as a function of time in the same manner. The sheets were immersed for 72 to 96 hours before this experiment. The sheets were kept at room temperature and were weighed on different time points until they reached their final (dry) values.

Soap loss was measured after immersion by drying the samples at 120° C. for 4 hours in vacuum. The soap loss varies with immersion time.

### Hydrogen Peroxide Release Testing Method

The release rate of hydrogen peroxide from hydrophilic silicone sheets is measured using a method developed by Hannig et al., Archives of Oral Biology 48, 559-566 (2003).

### Staining Procedure

To test the whitening efficacy, bovine teeth surfaces were prepared using a 600 grit paper and etched 30 sec each with 0.2M HCl, saturated Na₂CO₃, 1% phytic acid and finally rinsed with double distilled water. The samples were then stained artificially with a staining solution containing equal quantities of black tea and coffee. The teeth were immersed in staining solution for seven days at 37° C. with shaking at 110 rpm.

L*a*b* colorimetric measurements were performed for pre-stained, post stained and post treated samples with a CR-400 Chromameter. L* is a measure of response to the eye to lightness and darkness, a is a measure of redness and b is a measure of yellowness. The higher the L* value and the lower the b* value, the whiter teeth appear.

The whiteness index was calculated using the following equation:

Delta E=√[(Delta L*)²+(Delta a*)²+(Delta L*)²].

### Testing Sample 4.1

Here, we consider the experimental results on the water and the 6% hydrogen peroxide/water uptake and release processes in monolithic hydrophilic silicone sheets (shore 40 and shore 5). For this experiment 27% of sodium-olefin sulfonate C14-C16 (from Stephan) has been incorporated in the commercial elastomer Elastosil LR 3004 with a shore value of 40 hardness. The pure water and hydrogen peroxide/water uptakes of the cured sheet have been tested by immersion of hydrophilic test sheets with a known surface of about 4.4 cm diameter and a thickness of 100 μm and 500 μm, respectively, in demineralized water or hydrogen peroxide/water for a period of time.

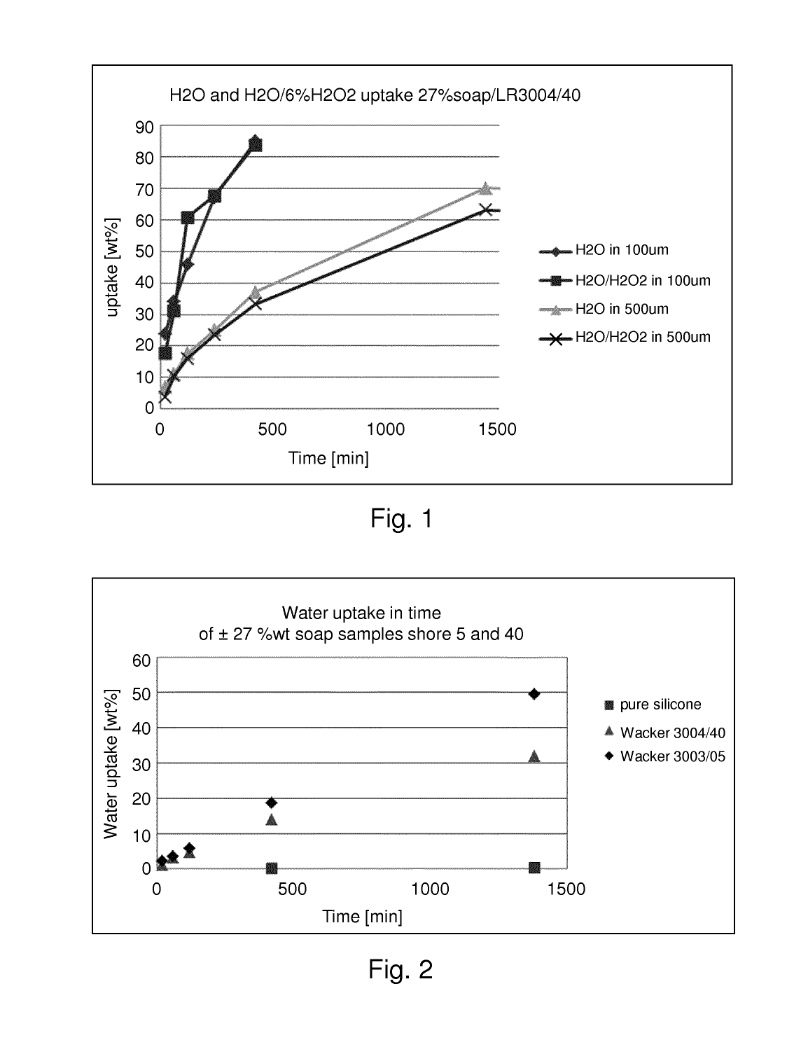

In FIG. 1 a graph is given showing the weight uptake (Y-axis) over time (X-axis) of pure water and hydrogen-peroxide/water in thermally cured hydrophilic silicone sheets (100 μm or 500 μm thickness and 40 Shore), as a function of soaking time.

Four lines are shown, each with measurement points indicated by geometrical shapes as follows:
diamonds(♦): H₂O uptake in sheet of 100 μm thickness;
squares (▪): H₂O₂/H₂O uptake in sheet of 100 μm thickness;
triangles (▴): H₂O uptake in sheet of 500 μm thickness;
crosses (x): H₂O₂/H₂O uptake in sheet of 500 μm thickness.

The water uptake and release can be adjusted by using a lower shore material. The difference in water uptake can be seen in FIG. 2, which is a graph similar to that of FIG. 1. Shown is the weight uptake (Y-axis) over time (X-axis) of pure water in thermally cured silicone sheets (1.2 mm thickness) shore 5 and 40 as a function of soaking time. The following legend applies:
squares (▪): non-modified silicone (reference);
triangles (▴): hydrophilically modified silicon; shore hardness of 40;
diamonds(♦):hydrophilically modified silicon; shore hardness of 45.

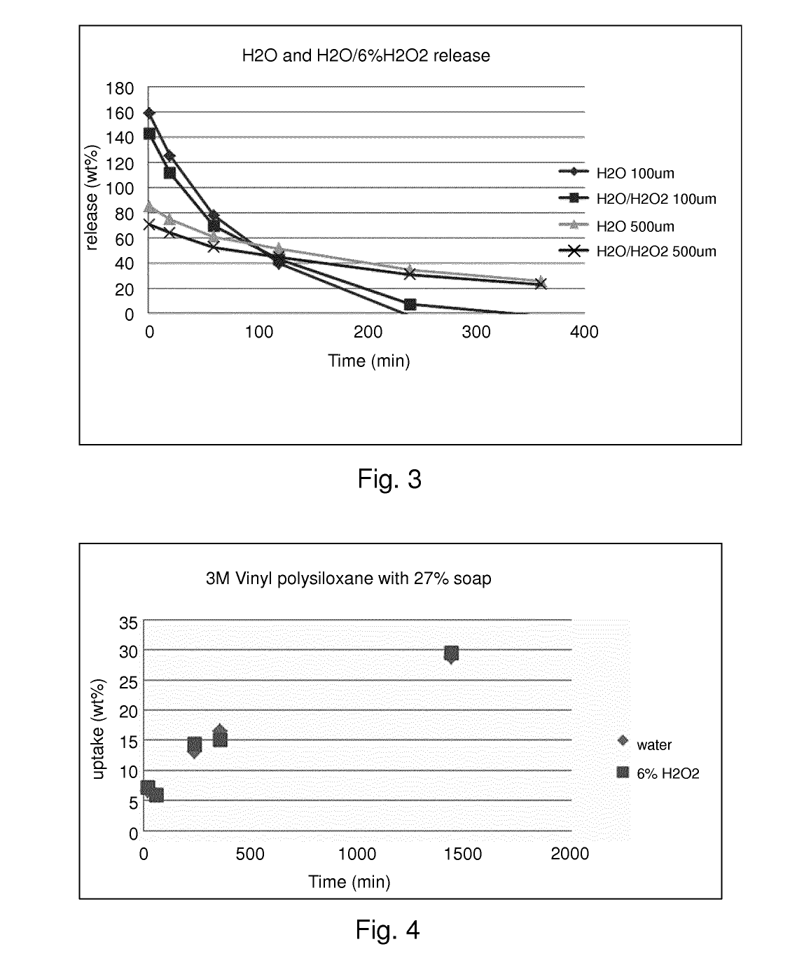

After completion of the uptake experiment, the silicone was taken out of the solution and left to dry in air (at room temperature). The results are shown in FIG. 3. This is a graph showing the release (weight loss; Y-axis) over time (X-axis), during drying at room temperature after uptake of pure water and hydrogen-peroxide/water in thermally cured hydrophilic silicone sheets (100 μm or 500 μm thickness, and 40 Shore) as a function of drying time.

FIG. 3 has the following legend:
diamonds(♦): release from H₂O soaked sheet of 100 μm thickness;
squares (▪): release from H₂O₂/H₂O-soaked sheet of 100 μm thickness;
triangles (▴):release from H₂O-soaked sheet of 500 μm thickness;
crosses (x): release from H₂O₂/H₂O-soaked sheet of 500 μm thickness.

From FIG. 3 it is clear that the uptake level and speed do not depend on the presence of the 6% H₂O₂ in the water. The speeds of uptake and release are however dependent on the thickness of the sheet (for a 100 μm sheet much faster than for a 500 μm sheet). The maximum weight uptake found is about 70% for the 500 μm sheet up to about 150% for 100 μm sheet (both for 27% soap additive). For 3% soap additive in the silicone, the maximum water weight uptake is found to be about 8% (for H₂O₂/water) and 13% (for water).

### Testing Sample 4.2

Here, we consider the experimental results on the water and 6% hydrogen peroxide/water uptake and release processes in the RT curable hydrophilic impression material.

Respectively 0% and 27% of sodium-olefin sulfonate C14-C16 (Stephan) have been incorporated in the commercial 3M Impression material (Imprint II, Vinyl Polysiloxane). The demineralized water and hydrogen-peroxide solution uptake in the hydrophilic impression material has been tested by full immersion of the sheets in the respective fluids for a period of time. Sheets were chosen here with surfaces of about 5.0 cm diameter and thicknesses of about 1.2 mm.

After 24 hours, the weight increase was about 30% (see FIG. 4). This does not dependent on whether the 6% hydrogen-peroxide solution or the demineralized water was added. A check measurement with this silicone material without soap additive showed no uptake of water. Therefore also in this case, the addition of soap in the silicone proves to be essential for creating water and agent storage capability in the silicone.

FIG. 4 is a graph showing the weight uptake (Y-axis) of demineralized water and hydrogen-peroxide/water in hydrophilic impression silicone sheets (about 1.2 mm thickness) as a function of soaking time (X-axis). Legend:
diamonds(♦):H₂O-soaked sheet;
squares (▪): H₂O₂/H₂O-soaked sheet.
   Hydrogen Peroxide Release from Silicone Sheets

### Release of 6% Hydrogen Peroxide

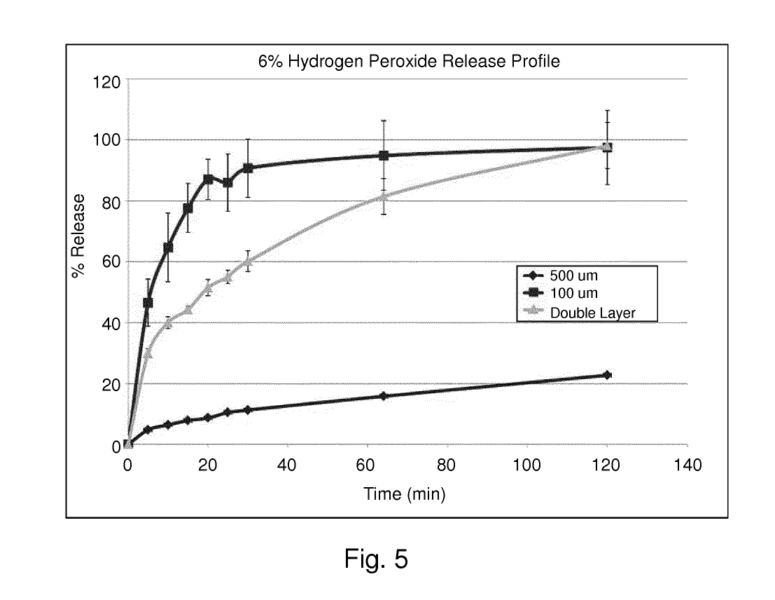

Using the Hannig detection method, the release of hydrogen peroxide from hydrophilic silicone sheets was measured with one side submerged in water. This was done after first fully loading the single (and double layer) sheets in a 6% hydrogen peroxide solution in water. Over the 120 minutes measuring time used here, a steady release of hydrogen peroxide is observed from the 500 μm sheet, reaching a release of 23% of the hydrogen peroxide content after 2 hours (see FIG. 5). This equates to a final hydrogen peroxide concentration of 0.008% obtained in the accepting water solution. The 100 μm sheet showed a rapid release of its hydrogen-peroxide loading within the first 20-25 minutes, releasing 87% within this time. This is a quite fast release process. The release rate then slowed over the next 95-100 minutes giving a final total release of 98% at the final reading after 120 minutes. The final hydrogen-peroxide concentration in the accepting water was 0.002%. The double-layered sheet demonstrated a slower rate of release than the 100 μm sheet, with a significantly lower percentage of its hydrogen-peroxide load released in the first 30 minutes (60% vs. 91% for the single 100 μm sheet). However, the rate of release was higher for the double layer sheet than for the 100 μm sheet in the 90 minutes that followed, giving an equal total release of 98% hydrogen peroxide at 120 minutes. This equates to a final hydrogen peroxide concentration in the receiving solution of 0.001%. Both the double-layered sheet and the 100 μm sheet curled during the release testing, this may have affected the release rates.

FIG. 5 is a graph showing the release of hydrogen peroxide into water from hydrophilic silicone sheets after presoaking in 6% hydrogen peroxide/water solution. Hydrophilic sheets with 27% soap additive of 100 μm thickness, and 500 μm thickness were used, or a double-layer sheet with 100 μm hydrophilic layer and 500 μm hydrophobic layer. One side of the hydrophilic layer was submerged in the testing water fluid for release. Legend:
squares (▪):layer of 100 μm thickness;
diamonds(♦):layer of 500 μm thickness;
triangles (▴):100 μm hydrophilic+500 μm hydrophobic silicone layers.

Overall the 500 μm sheet gave the slowest rate of release by percentage hydrogen-peroxide content, but after 120 minutes it already released 5-10 times larger total volume of hydrogen-peroxide than the (much thinner) sheets by virtue of it being able to absorb a larger absolute quantity of 6% hydrogen peroxide initially. The double-layered sheet and the 100 μm sheet both release essentially their entire hydrogen-peroxide load within the 120 minutes with a very similar overall release rate; however a faster initial rate is achieved by the 100 μm sheet. The sheets floated on the surface of the water during the release tests and so the diffusion of hydrogen peroxide can be though to involve one face of the sheet only.

### 0.1% Hydrogen Peroxide Release

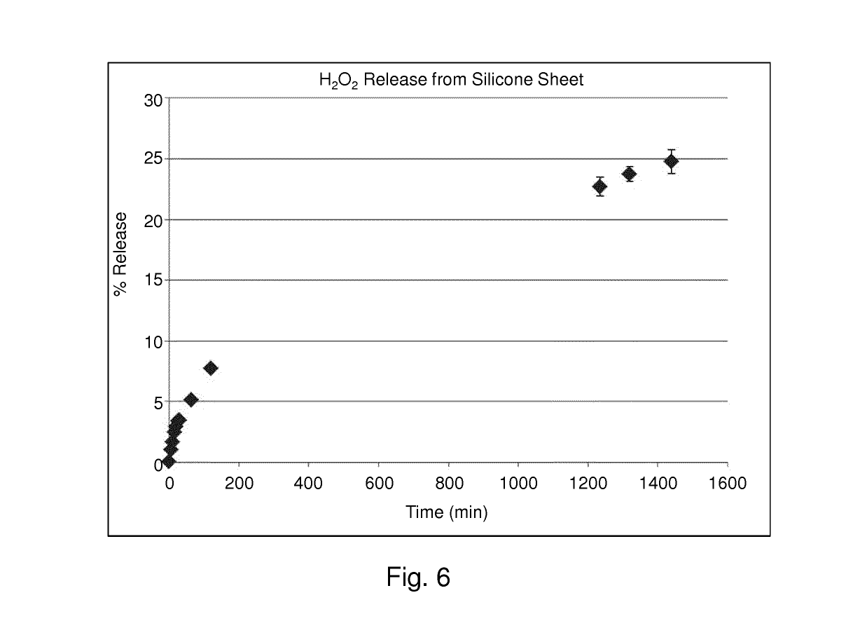

In FIG. 6, the result is shown of the release of hydrogen-peroxide from silicone to water, after fully pre-soaking of a 500 μm thick hydrophilic silicone sheet. The figure shows the average of three experiments. Also at this low concentration of hydrogen-peroxide, a continuous release of hydrogen-peroxide is found up to the maximum 24 hours of time of this experiment. So, a steady release of a low concentration from hydrophilic silicone is feasible, for example for use in anti-bacterial applications.

FIG. 6. is a graph showing the release (Y-axis) over time (X-axis) of hydrogen peroxide into water from hydrophilic silicone sheets after presoaking in 0.1% hydrogen peroxide/water solution. A hydrophilic sheet with 27% soap additive of 500 μm thickness was used. One side of the hydrophobic layer was submerged in the testing water fluid for release.

Tooth Whitening with Silicone Sheets on Bovine

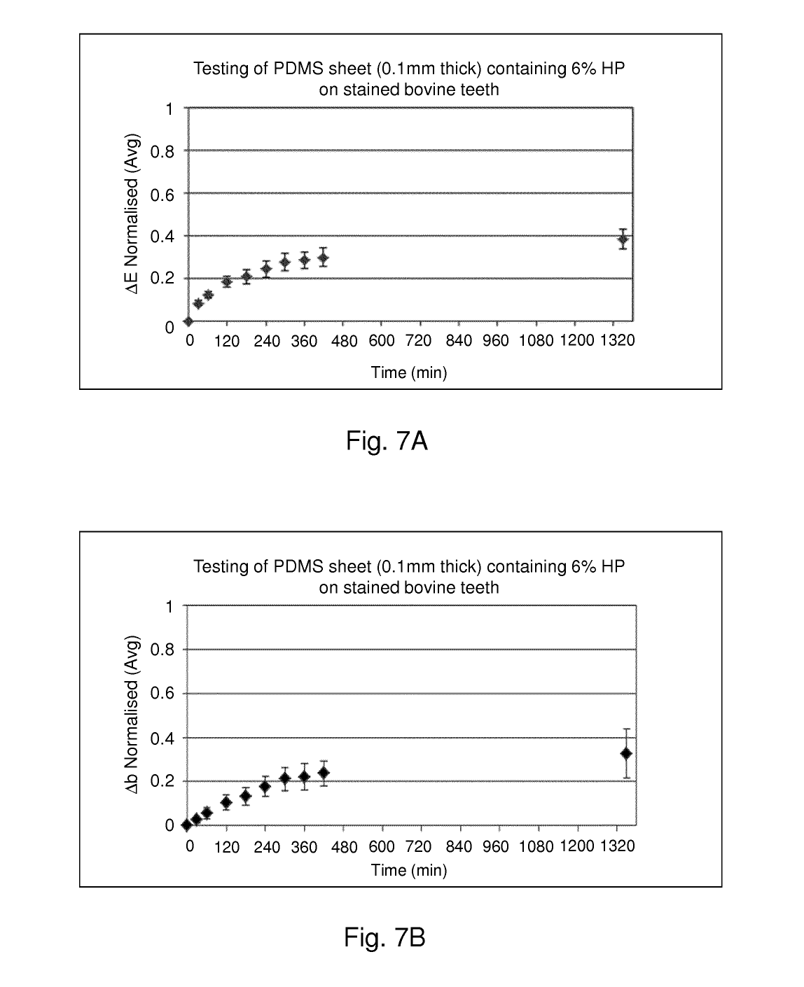

The following experiment was carried out to evaluate the whitening efficacy of PDMS sheets pre-loaded in 6% hydrogen-peroxide. The hydrophilic PDMS sheets of 0.1 mm thick containing 27% soap was cut into appropriate size (1.5 cm×1.5 cm) and soaked in a 6% hydrogen-peroxide solution overnight. The following day, soaked individual sheets were placed on stained teeth, covered with a cover slip on top of each tooth for better adhesion of the sheet to the tooth. The teeth samples were kept at 21° C. in a humid environment for treatment. The L*, a* and b* measurements were performed after 30 and 60 minutes of treatment and then after every one hour treatment. The teeth were then treated overnight for 15 hours and measured L*, a* and b* values on the next day. To observe any drying effect, the teeth were immersed in phosphate buffered saline for 50 minutes after the overnight treatment procedure, followed by a re-measurement of the L*, a* and b* values. Here, a ΔE of 1 was found due to the PBS treatment. The whitening results shown in FIG. 7 were corrected for this influence of dehydration.

FIG. 7 are graphs showing the results (ΔE—FIG. 7a and Δb—FIG. 7b) of bovine tooth whitening test with 0.1 mm hydrophilic silicone sheets preloaded in 6% hydrogen peroxide/water solution.

The ΔE and Δb data were normalized to the pre-stain value of each individual tooth sample before taking the average of samples. A normalized value of 1 represents the complete removal of the added stain from the teeth, ΔE of ˜20 in this case. The overall color change is represented by ΔE. It can be concluded from the above results that the whitening of the samples was slow and reached 0.3 relative units (corresponding with ΔE=6) after 7 hour treatment period. An over-night treatment period of 15 hours did not make any further significant difference in ΔE value. The Δb value represents a change in the color of the teeth from yellow to blue, and from above chart it can be observed that the yellowness of the tooth was lowering by 0.25 relative units after 7 hour treatment period.

Tooth Whitening with Silicone Trays on Bovine

Further to the testing of silicone sheets the whitening efficacy of a hydrophobic silicone tray containing an inlay of 3M hydrophilic material was tested. To observe any potential influence of the positioning of the tooth in the tray, the tooth samples were placed in the array holder in different orientation such as facing down or facing towards one of both sides of the tray.

The tray was then soaked in 6% hydrogen-peroxide solution for two days for complete saturation of the tray hydrophilic inlay with 6% hydrogen-peroxide. The whitening experiment was performed by capping an array of stained teeth with the tray. The dehydration of teeth was reduced by in a way to avoid dehydration as much as possible by covering the silicone tray with wet tissue and keeping the entire system in a sealed plastic bag for the over-night treatment, followed by the immersion in PBS for three hours to rehydrate the teeth samples completely. As a comparison, a control experiment was performed by whitening a set of teeth by submerging them in a 6% hydrogen peroxide solution.

The treatment was carried over six hours with L*a*b* values measured every hour and then continued overnight for 14 hours and measured L*a*b* values the following day. The same procedure was repeated for the direct 6% hydrogen peroxide solution experiment however the teeth were left on the bench top for treatments rather than the 37° C. used for the silicone tray. After the overnight treatment, the samples from the silicone tray and from the 6% hydrogen peroxide solution were immersed in PBS for three hours and we re-measured the L*a*b* values to evaluate the rehydration effect of the teeth. It was observed that an average of two units difference in ΔE values was observed after three hours of PBS immersion.

These whitening results are obtained after averaging over three tooth samples. The whitening results were corrected for the de-hydration influences.

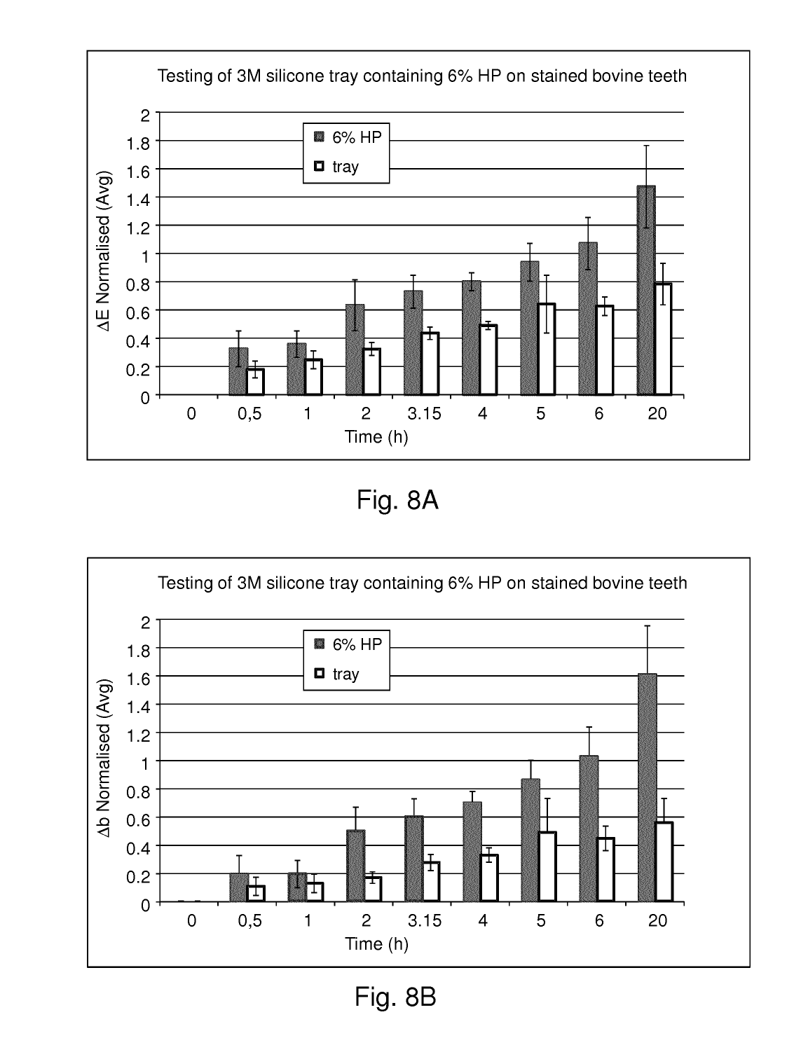

FIG. 8a is a graph representing the results of a bovine tooth whitening test showing the normalized ΔE (Y-axis) as a function of total treatment time (X-axis) obtained with a hydrophilic 3M silicone filled tray (white bars) and with the control experiment in 6% hydrogen peroxide solution (dark bars).

It can be observed that the silicone tray is releasing the hydrogen peroxide at about 2× slower rate compared to the control experiment. However, it should be referred that the volume of the 6% hydrogen peroxide absorbed by the silicone tray was not measured quantitatively after immersing it in 6% hydrogen peroxide solution for two days. Note however that in other soaking experiments we measured that 3M hydrophilic silicone sheets are taking up about 30 wt. % of hydrogen-peroxide/water solution after 24 hours. Therefore the availability of hydrogen peroxide from the silicone tray to the teeth might have been more limited compared to the case of a full immersion directly in 6% hydrogen peroxide solution. The samples treated with the silicone tray showed an absolute ΔE of about 2-3 units after an hour treatment period. This value increased progressively over the six hours treatment period.

The yellowness of the teeth is represented by b* values and Δb values were also plotted to show the change in the yellowness of the teeth after treatment period. After 6 hours treatment period, the 6% hydrogen peroxide solution removed the entire added yellow color from the staining solution. The silicone tray removed less than half of the added color by that time. The corresponding absolute value of Δb for the tray after 6 hours is −5.

FIG. 8b is a graph representing the results of a bovine tooth whitening test showing the normalized Δb (Y-axis) as a function of total treatment time (X-axis) obtained with a hydrophilic 3M silicone filled tray (white bars) and with the control experiment in 6% hydrogen peroxide solution (dark bars).

Fluoride Release from Silicone

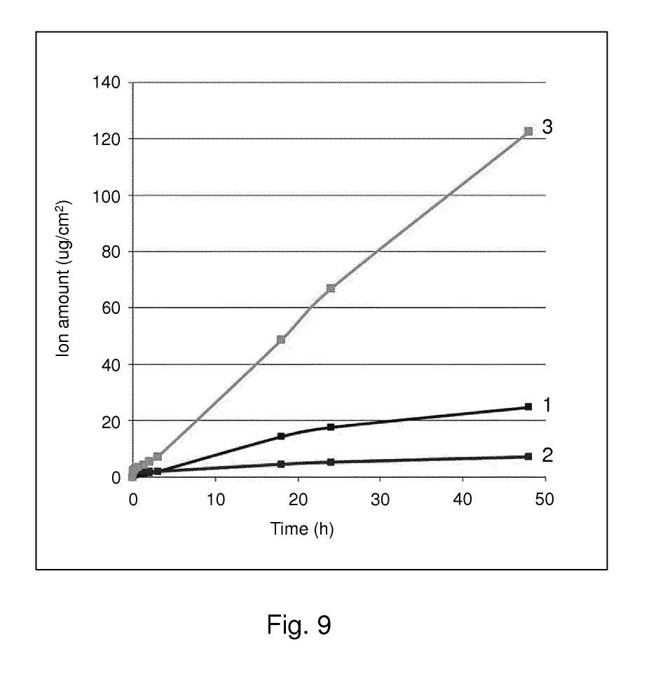

Initial experiments were performed to explore the options for a slow release of fluoride from silicone. For these tests, 1% of NaF (crystals) were added to the standard hydrophilic silicone (Shore 40, with 27% soap), cured at 135° C. The resulting silicone samples were immerged in demi-water. Ion chromatography was used to measure the release of the various components from the silicone into the demi-water as a function of time (see FIG. 9).

FIG. 9. is a graph showing the ion release (Y-axis) from standard hydrophilic silicone with NaF additive to demi-water, as a function of the time of immersion (X-axis). Legend:
Line 1: fluoride;
Line 2: chloride:
Line 3: sulphate.

A continuous release of F⁻ from the silicone is measured. This release is sufficient to sustain a steady-state F⁻ concentration level of about 1 ppm. The measured Cl⁻ and SO₄²⁻ originate from the soap additive in the silicone.

1. A composition for delivering an oral care substance to the oral cavity, said composition comprising at least one oral care agent contained in an elastomeric polysiloxane matrix, said matrix being a hydrophilic polysiloxane comprising a network based on repeating units of hydrophobic siloxane monomers and units of siloxane monomers comprising one or more hydrophilic side groups, wherein the hydrophilic siloxane units comprise a bound residue of one or more alpha-olefinic soap monomers.

2. A composition according to claim 1, wherein the alpha-olefinic soap is an alpha-olefin sulfonate having a carbon chain length of from 3 to 28, preferably 10 to 18.

3. A composition according claim 1, further comprising one or more unreacted alpha-olefinic soap monomers.

4. A composition according to claim 1, wherein the total weight of alpha-olefinic soap monomers in the composition is 10-30% relative to the total weight of the siloxane units in the composition.

5. A composition according to claim 1, wherein the hydrophobic siloxane units are selected from dialkyl siloxane, diaryl siloxane, and mixtures thereof.

6. A composition according to claim 1, wherein the hydrophilic polysiloxane comprises a network of a siloxane prepolymer comprising vinyl groups subjected to the platinum salt-induced crosslinking with a siloxane component comprising Si—H groups.

7. A composition according to claim 1, wherein the oral care agent is selected from the group consisting of bleaching agents, demineralization agents, desensitizing agents, antibacterial agents, anti biofilm agents and mixtures thereof.

8. A composition according to claim 7, wherein the oral care agent is hydrogen peroxide.

9. A method of making a composition for delivering an oral care substance to the oral cavity, the method comprising the steps of
(i) providing a hydrophilic polysiloxane capable of taking up more than 10% by weight, preferably more than 15% by weight, of water and up to 500% by weight of water, after immersion in demineralized water at room temperature for a sufficient time to reach saturation;
(ii) providing an aqueous liquid comprising an oral care agent dissolved or dispersed therein;
(iii) soaking the aqueous liquid into the hydrophilic polysiloxane, wherein the hydrophilic polysiloxane is a polysiloxane as defined in claim 1.

10. The use of an elastomeric polysiloxane as defined in claim 1, as a carrier for an oral care substance., particularly for a dental bleaching agent.

11. A method for delivering an oral care substance to the oral cavity of a subject, preferably a human subject, said substance being administered to the oral cavity in a polymer matrix, wherein the polymer is an elastomeric polysiloxane as defined in claim 1.

12. A shaped article comprising a layer of hydrophobic polysiloxane elastomer molded against a layer of a hydrophilic polysiloxane elastomer, said hydrophilic polysiloxane being as defined in claim 1 wherein the hydrophobic polysiloxane determines one outer surface of the device and the hydrophilic polysiloxane determines another outer surface of the device.

13. A dental tray comprising a material shaped according to a dental impression and comprising an oral care active substance to be released therefrom, wherein said material comprises a hydrophilic polysiloxane as defined in claim 1.

14. A kit for the local administration of an oral care agent to the teeth of a subject, the kit comprising
a dental tray made of a silicone elastomer;
a curable hydrophilic silicone material;
an aqueous liquid comprising an oral care agent;
wherein the curable hydrophilic silicone material comprises a siloxane prepolymer component comprising vinyl groups and a siloxane crosslinker component comprising Si—H groups, wherein either or both of the components comprise an alpha-olefinic soap.

15. A kit according to claim 14, wherein the kit is used to create the oral care agent for delivery to the teeth of the subject.
